(19) United States
(12) Patent Application Publication
(10) Pub. No.: US 2018/0250153 A1
(43) Pub. Date: Sep. 6, 2018

(54) SPLINT

(71) Applicant: Lee Kleynhans, Trentham (NZ)

(72) Inventor: Lee Kleynhans, Trentham (NZ)

(21) Appl. No.: 15/910,627

(22) Filed: Mar. 2, 2018

(30) Foreign Application Priority Data
Mar. 3, 2017 (NZ) 729715

(51) Int. Cl. A61F 5/058

(52) U.S. Cl. CPC A61F 5/05875

(57) ABSTRACT

In a preferred embodiment, the invention is a splint for use in straightening the finger of a person. It has a first cradle for receiving a part of a finger, and a second cradle for receiving another part of a finger and a screw. The first and second cradles are pivotally connected and secured to the finger. The screw is attached to a cradle and arranged so as to freely abut against a surface on the opposing cradle. The screw can be moved so as to change the angle of the finger correspondingly.

### CROSS-REFERENCE TO RELATED APPLICATIONS

This application claims the benefit of New Zealand Patent No. 729715, filed on Mar. 3, 2017. The entire disclosure of the above application is hereby incorporated herein by reference.

### FIELD

A preferred form of the invention relates to a splint for use in straightening the finger or another digit of a person.

### BACKGROUND

The statements in this section merely provide background information related to the present disclosure and may not constitute prior art.

In the context of applying medical treatment to humans it is sometimes necessary to straighten a curled or hooked finger before it can be operated on, for example in the case of proximal interphalangeal joint flexion contractures. The process is typically done in stages. First the finger is straightened a little and a plaster cast is applied to hold it there. After a period of time in the cast the finger will have been trained into this ‘more straightened’ disposition. The cast is then removed, the finger straightened a little more, and a new plaster cast applied to hold it there. The process is repeated as many times as necessary until the finger is sufficiently straightened to enable a surgeon to more easily operate on it. This can be an inefficient and expensive process as a patient may need to visit a clinic and receive attention from medical professionals each time a new cast is applied. A further problem is that plaster casts can be uncomfortable as they enclose the skin and can cause itching and skin damage. It is an object of a preferred embodiment of the invention to go at least some way towards addressing one or more of these problems. While this object applies to the preferred embodiment, it should not be seen as a limit on the scope of the claims. The object of the invention per se is simply to provide the public with a useful choice.

The term “comprising”, when used in this document in relation to a combination of features, should not be taken to exclude the option of there being further features. The term should therefore be interpreted in a non-limiting way.

### SUMMARY

According to an aspect of the invention there is provided a splint comprising:

a first section proportioned for receiving part of a digit;

a second section proportioned for receiving another part of the digit comprising a contact surface; and

an adjuster;

the first and second sections having means for securing them to the digit and the adjuster being such that it freely abuts or butts against the contact surface and can be operated to push against the contact surface to angle adjust the first and second sections so that the angle of the digit changes correspondingly.

Optionally the first and second sections are pivotally connected. Optionally the first and second sections each comprise a cradle.

Optionally each cradle has a flexible strap suitable for holding the cradle to the digit.

Optionally each cradle has a pair of upper slots, one each side of the cradle, and the strap extends through the slots to enable the cradle to be secured to the digit.

Optionally the adjuster is supported by one of the sections and can be moved to push the other section to angle adjust the two.

Optionally the adjuster comprises a threaded member that can be turned to push on and pivot one of the sections with respect to the other.

Optionally the threaded member is located in a bore of one of the sections.

Optionally the threaded member comprises a screw which passes through a nut.

Optionally the first and second sections are pivotally connected by way of a stud and recess connection, the stud being located and able to turn in the recess.

According to a further aspect of the invention there is provided a method for straightening a digit (a finger, thumb or toe), comprising applying a splint as above to the digit and in stepwise fashion altering the angle between the two sections by way of the adjuster to train the digit to a straightened or more straightened disposition.

Further areas of applicability will become apparent from the description provided herein. It should be understood that the description and specific examples are intended for purposes of illustration only and are not intended to limit the scope of the present disclosure.

### DRAWINGS

Some preferred embodiments of the invention will now be described by way of example and with reference to the accompanying drawings, of which:

FIG. 1 is an isometric view of a finger splint when in use;

FIG. 2 is a plan view of the splint in use;

FIG. 3 is an isometric view showing details of the splint;

FIG. 4 is an isometric view of cradles forming part of the splint;

FIG. 5 is a side view of the cradles plus an adjuster screw;

FIG. 6 is an isometric view of one of the cradles; and

FIG. 7 is an isometric view of the other cradle.

### DETAILED DESCRIPTION

The following description is merely exemplary in nature and is not intended to limit the present disclosure, application, or uses. It should be understood that throughout the drawings, corresponding reference numerals indicate like or corresponding parts and features.

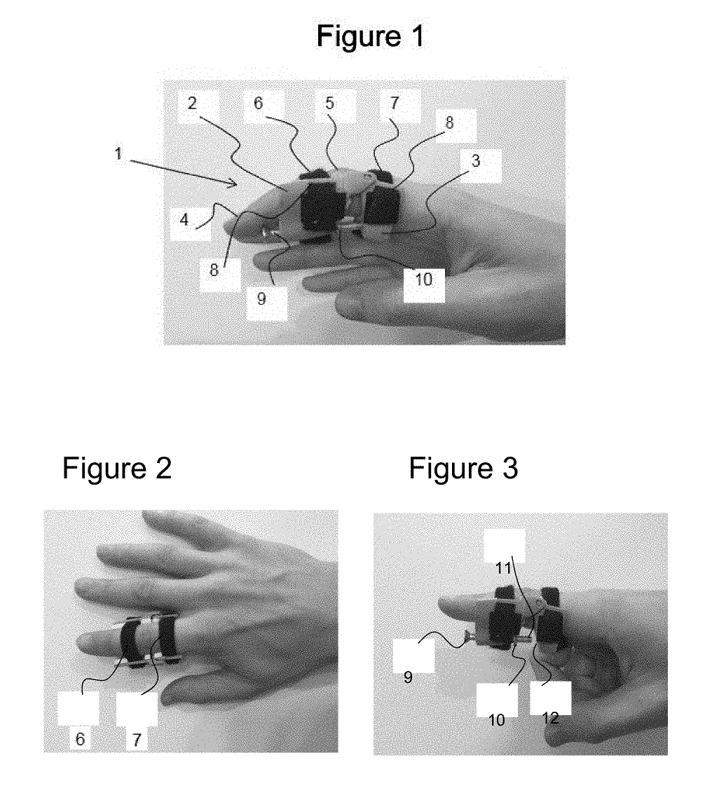

Referring to FIGS. 1 and 2, the splint 1 comprises a first cradle 2 and a second cradle 3 in hinged relationship. As shown, the cradles receive forward and rear parts of a finger 4, either side of a bone joint 5, and are secured to the finger by way of hook and loop (e.g., Velcro™ hook-and-loop fastener) strips 6, 7. The strips are threaded through slots 8 either side of each cradle. In the instance shown the finger is slightly curled or hooked and needs to be straightened prior to surgery.

With reference to FIGS. 1, 3, 5 and 7, the lower part of the first cradle 2 has a bore 2a (see FIG. 7). A threaded screw 9 passes through the bore and a nut 10 at the forward end of the bore (see FIGS. 1 and 3). The nut 10 fits snugly against the first cradle 2 so that it cannot turn with the screw 9.

Referring to FIG. 5, the flat forward end 11 of the screw abuts or butts against a forward contact surface 12 of the second cradle 3. By hand turning the screw so that it moves forward its flat end pushes against the surface 12 to increase the angle between the two cradles. This has the effect of straightening the finger. The process can be achieved in stages over several days or weeks until the finger has been trained to a substantially straightened disposition, ready for surgery. The angle between the cradles, and therefore of the finger, can be reduced by turning the screw the opposite way. In some embodiments a screwdriver may be used to turn the screw 9, but hand turning is preferred as it is simpler.

FIG. 3 illustrates detail of the splint with the finger is in a straight disposition and the screw 9 positioned just short of the contact surface 12. To retain the finger in a straight disposition the screw 9 would of course be turned to move forward and abut or butt against the contact surface 12.

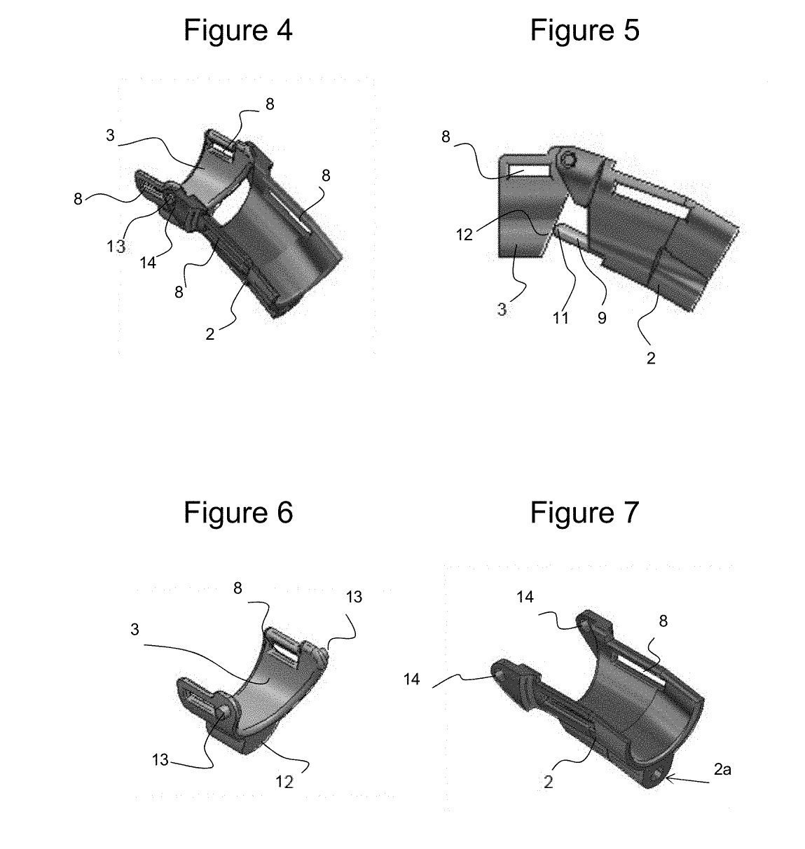

Referring to FIGS. 4-7, the hinge connection between the two cradles is achieved by inserting side 13 studs of the second cradle into side apertures 14 of the first cradle. The resilience of the material from which the cradles are formed helps maintain the connection. For example during assembly the upper edges of the second cradle can be squeezed together slightly to align the studs 13 with the side apertures of the first cradle 14, and then released. On release the studs 13 press outwardly into the apertures 14 to provide a tension fit. In some embodiments the apertures 14 may not pass all the way through the cradle; they may comprise recesses.

The splint can be produced from any suitable material or materials. The cradles may for example be of a suitable coated plastic material and formed by 3D printing. They may be subsequently coated with resin. In some cases padding may be added to the cradles to cushion the finger. Production by 3D printing enables the splint to be readily customized for the patient concerned. In this regard the dimensions of the cradles can be quickly set using computer software prior to printing. While 3D printing in plastic is preferred (e.g., using nylon, polyamide, ABS or PLA materials), in other embodiments the cradles may be formed from carbon fiber, metallic or other suitable materials. The screw and nut may be of stainless steel. The splint may also be sized for use with other digits, namely a thumb or toe.

While some preferred embodiments of the invention have been described by way of example it should be appreciated that modifications and improvements can occur without departing from the scope of the following claims.

What is claimed is:

1. A splint comprising:
a first section proportioned for receiving part of a digit;
a second section proportioned for receiving another part of the digit comprising a contact surface; and
an adjuster;
the first and second sections having means for securing them to the digit and the adjuster being such that it freely abuts against the contact surface and can be operated to push against the contact surface to angle adjust the first and second sections so that the angle of the digit changes correspondingly.

2. The splint according to claim 1, wherein the first and second sections are pivotally connected.

3. The splint according to claim 1, wherein the first and second sections each comprise a cradle.

4. The splint according to claim 3, wherein each cradle has a flexible strap suitable for holding the cradle to the digit.

5. The splint according to claim 4, wherein each cradle has a pair of upper slots, one each side of the cradle, and the strap extends through the slots to enable the cradle to be secured to the digit.

6. The splint according to claim 1, wherein the adjuster is supported by one of the sections and can be moved to push the other section to angle adjust the sections.

7. The splint according to claim 6, wherein the adjuster comprises a threaded member that can be turned to push on and pivot one of the sections with respect to the other.

8. The splint according to claim 7, wherein the threaded member is located in a bore of one of the sections.

9. The splint according to claim 7, wherein the threaded member comprises a screw which passes through a nut.

10. The splint according to claim 1, wherein the first and second sections are pivotally connected by way of a stud and recess connection, the stud being located and able to turn in the recess.

11. The splint according to claim 1, wherein:
a) the first and second portions are each in the form of a cradle;
b) the cradles have side openings which receive straps suitable for fastening the splint to a finger or thumb;
c) one of the cradles has a bore containing a threaded member that can be turned to push against a contact surface of the other cradle to angle adjust the cradles; and
d) the cradles engage in a hinge arrangement by way of a resilient stud and aperture/recess fit.
